(19) United States
(12) Patent Application Publication
(10) Pub. No.: US 2014/0322151 A1
(43) Pub. Date: Oct. 30, 2014

(54) Use Of Isosorbide Caprylates/Caprates In Deodorants And Antiperspirants

(71) Applicant: CLARIANT INTERNATIONAL LTD, Muttenz (CH)

(72) Inventors: Tom Fricke, Liederbach (DE); Peter Klug, Grossostheim (DE); Carina Mildner, Frankfurt am Main (DE)

(73) Assignee: CLARIANT FINANCE (BVI) LIMITED, Tortola (VG)

(21) Appl. No.: 14/358,449

(22) PCT Filed: Nov. 21, 2012
(86) PCT No.: PCT/EP2012/004827

§ 371 Date: May 15, 2014

(30) Foreign Application Priority Data
Nov. 22, 2011 (DE) 10 2011 119 033.7

(51) Int. Cl. A61K 8/49; A61K 8/36; A61K 8/60; A61Q 15/00

(52) U.S. Cl. CPC A61K 8/4973; A61Q 15/00; A61K 8/361; A61K 8/36; A61K 8/60; A61K 8/4926
USPC 424/67; 424/65

(57) ABSTRACT

Disclosed is the use of compositions containing one or more isosorbide capryiates/caprates in antiperspirants and deodorants for improving the action thereof in reducing body odor. The improvement of the action of the antiperspirants and deodorants relates especially to the intensity of reducing body odor and/or to the duration of reducing body odor.

The present invention relates to the use of isosorbide caprylates/caprates in antiperspirants and deodorants for improving the action thereof in reducing body odor.

Numerous active substances are known which can be used in antiperspirants and deodorants for combating body odor.

A disadvantage of using many of these active substances, however, is that their preparation is often complex and based on synthetic raw materials. For some of these active substances, such as e.g. for aluminum-containing substances, moreover, alternatives are sought for toxicological reasons. Furthermore, the action of the active substances against body odor is often in need of improvement, meaning that high use concentrations are necessary for an adequate action. From the point of view of consumers, antiperspirants and deodorants with a long-lasting action in excess of 24 hours are increasingly being demanded.

It was therefore the object to provide compounds which improve the action of antiperspirants and deodorants. Moreover, they should be based on renewable raw materials and be toxicologically acceptable.

Surprisingly, it has now been found that this object is achieved by compositions comprising one or more isosorbide caprylates/caprates.

The invention therefore provides the use of a composition comprising one or more isosorbide caprylates/caprates (composition A) in antiperspirants and deodorants for improving the action thereof in reducing body odor.

Isosorbide caprylates/caprates are understood as meaning both the pure caprylates or caprates, as well as mixtures of caprylates and caprates.

In a preferred embodiment of the invention, the isosorbide caprylates/caprates are mixtures of caprylates and caprates preferably in the molar ratio of 1:2 to 2:1 and particularly preferably in the molar ratio of 2:3 to 3:2.

In a further preferred embodiment of the invention, the isosorbide caprylates/caprates are caprylates with a fraction of the caprates of at most 5 mol %, particularly preferably of at most 0.5 mol % and particularly preferably pure caprylates which are free from caprates.

In a further preferred embodiment of the invention, the isosorbide caprylates/caprates are caprates with a fraction of the caprylates of at most 5 mol %, particularly preferably of at most 0.5 mol % and especially preferably pure caprates which are free from caprylates.

Body odor is preferably understood as meaning perspiration odor.

In a further preferred embodiment of the invention, the compositions A comprise, besides the one or more isosorbide caprylates/caprates, additionally one or more sorbitan caprylates/caprates.

In a preferred embodiment of the invention, the sorbitan caprylates/caprates are mixtures of caprylates and caprates preferably in the molar ratio of 1:2 to 2:1 and particularly preferably in the molar ratio of 2:3 to 3:2.

In a further preferred embodiment of the invention, the sorbitan caprylates/caprates are caprylates with a fraction of the caprates of at most 5 mol %, particularly preferably of at most 0.5 mol % and especially preferably pure caprylates which are free from caprates.

In a further preferred embodiment of the invention, the sorbitan caprylates/caprates are caprates with a fraction of the caprylates of at most 5 mol %, particularly preferably of at most 0.5 mol % and especially preferably pure caprates which are free from caprylates.

The isosorbide caprylates/caprates may be isosorbide monocaprylate/caprate, isosorbide dicaprylate/caprate or any desired mixtures thereof. The sorbitan caprylates/caprates may be mono-, di-, tri- or tetracaprylate/caprate or any desired mixtures thereof.

Sorbitan caprylates/caprates and isosorbide caprylates/caprates are based on renewable raw materials and are toxicologically acceptable.

The use of compounds such as isosorbide caprylates/caprates and/or sorbitan caprylates/caprates in compositions such as e.g. in cosmetic, dermatological or pharmaceutical compositions, is already known.

DE 10 2009 022 444 (Clariant) describes liquid compositions comprising sorbitan monocaprylate and antimicrobial active ingredients such as e.g. specific organic acids and their salts, specific formaldehyde donors, specific isothiazolinones, specific paraben esters and their salts and specific pyridones and their salts, as well as their use for preserving cosmetic, dermatological or pharmaceutical products.

DE 10 2009 022 445 (Clariant) discloses liquid compositions comprising sorbitan monocaprylate and alcohol and their use for preserving cosmetic, dermatological or pharmaceutical products.

WO 2010108738 A2 (Evonik) describes formulations for the cleaning and care of human or animal body parts, comprising sorbitan carboxylic acid esters, where the carboxylic acid moiety of the sorbitan carboxylic acid ester is derived from a carboxylic acid comprising 6 to 10 carbon atoms and the sorbitan carboxylic acid esters have a hydroxyl number (OH number) of greater than 350, and also the use of said sorbitan carboxylic acid esters as viscosity regulators, care active ingredient, foam booster or solubilizer in cleaning or care formulations.

JP 8173787 (A) (Lion) describes a composition comprising a surface-active substance comprising a fatty acid ester of dehydrated sorbitol and the use as oil-in-water emulsifier and as cleaning base. The compositions can comprise mono- or diesters of caprylic acid and/or capric acid with a polyol selected from the group consisting of 1,5-sorbitan, A-sorbitan and isosorbide.

Isosorbide caprylates/caprates and sorbitan caprylates/caprates can be prepared e.g. by methods known to the person skilled in the art. For example, these compounds can be prepared by esterification of sorbitol or isosorbide according to customary methods known to the person skilled in the art, with both sorbitol and isosorbide themselves as well as the acid components used for the esterification in turn being commercially available.

By virtue of the use according to the invention, the action of the antiperspirants and deodorants is preferably improved as regards the extent of the reduction in body odor.

By virtue of the use according to the invention, the action of the antiperspirants and deodorants is furthermore preferably improved as regards the duration of the reduction in body odor.

Besides the one or more isosorbide caprylates/caprates, the composition A preferably comprises one or more further compounds selected from the group consisting of caprylic acid, capric acid, sorbitol, sorbitol caprylates, sorbitol caprates, sorbitan and isosorbide and optionally one or more sorbitan caprylates/caprates.

The sorbitol caprylates and sorbitol caprates may be the corresponding mono-, di-, tri-, tetra-, penta- and hexaesters of sorbitol or any desired mixtures of these substances.

Furthermore preferably, the total amount in the composition A of the one or more isosorbide caprylates/caprates and the optionally one or more sorbitan caprylates/caprates additionally present in the composition A is at least 50.0% by weight, preferably at least 60.0% by weight, particularly preferably at least 70.0% by weight and especially preferably at least 75.0% by weight, based on the total weight of the composition A.

Furthermore, the composition A preferably comprises isosorbide monocaprylate/caprate.

Besides isosorbide monocaprylate/caprate, the composition A furthermore preferably additionally comprises sorbitan monocaprylate/caprate.

Particularly preferably the total amount in the composition A of isosorbide monocaprylate/caprate and the sorbitan monocaprylate/caprate optionally additionally present in the composition A is at least 30.0% by weight, preferably at least 35.0% by weight, particularly preferably at least 40.0% by weight and especially preferably at least 45% by weight, in the composition A based on the total weight of the composition A. Here, the total amount of isosorbide monocaprylate/caprate and the sorbitan monocaprylate/caprate optionally additionally present in the composition A can be up to 100% by weight, in a preferred embodiment of the invention, the total amount in the composition A of isosorbide monocaprylate/caprate and the sorbitan monocaprylate/caprate optionally additionally present in the composition A, however, is only up to 90.0% by weight, preferably up to 80.0% by weight and particularly preferably up to 70.0% by weight.

Further particularly preferably, the composition A additionally comprises sorbitan monocaprylate/caprate as well as isosorbide monocaprylate/caprate and the weight ratio of sorbitan monocaprylate/caprate to isosorbide monocaprylate/caprate in the composition A is from 20:1 to 1:100, preferably from 10:1 to 1:10, particularly preferably from 6:1 to 1:6, especially preferably from 3:1 to 1:5 and extraordinarily preferably from 1:1 to 1:4.

In a preferred embodiment of the invention, the compositions A comprise either no caprylic acid and capric acid or up to 1.0% by weight of caprylic acid and capric acid.

In a further preferred embodiment of the invention, the OH number of the mixture present in the composition A of the one or more isosorbide caprylates/caprates, the optionally one or more sorbitan caprylates/caprates additionally present therein and the optionally one or more compounds additionally present therein selected from the group consisting of caprylic, acid, capric acid, sorbitol, sorbitol caprylates, sorbitol caprates, sorbitan and isosorbide or of the composition A consisting of this mixture is less than or equal to 460, preferably less than or equal to 390, particularly preferably less than or equal to 340, especially preferably less than or equal to 260 and extraordinarily preferably less than or equal to 225.

in a further preferred embodiment of the invention, the compositions A comprise no compounds selected from sorbitol, sorbitol caprylates and sorbitol caprates.

In a further preferred embodiment of the invention, the compositions A consist of the one or more isosorbide caprylates/caprates, optionally additionally the one or more sorbitan caprylates/caprates and optionally additionally the one or more compounds selected from the group consisting of caprylic, acid, capric acid, sorbitol, sorbitol caprylates, sorbitol caprates, sorbitan and isosorbide.

If the compositions A comprise one or more compounds selected from sorbitol, sorbitol caprylates and sorbitol caprates, these compounds are present together in the compositions A preferably in an amount less than or equal to 5.0% by weight, particularly preferably in an amount less than or equal to 3,0% by weight, particularly preferably in an amount less than or equal to 1.0% by weight and extraordinarily preferably in an amount less than or equal to 0.5% by weight, where the data in % by weight are in each case based on the total weight of the finished compositions A.

The hydroxyl number or OH number of a substance is understood as meaning the amount of KOH in mg which is equivalent to the amount of acetic acid bonded during the acetylation of 1 g of substance.

Suitable determination methods for ascertaining the OH number are e.g. DGF C-V 17 a (53), Ph. Eur. 2.5.3 Method A and DIN 53240.

In the context of the present invention, the OH numbers are determined in accordance with DIN 53240-2. The procedure here is as follows: 1 g of the homogenized sample to be measured is weighed in to 0.1 mg precisely. 20.00 ml of acetylation mixture (acetylation mixture: 50 ml of acetic anhydride are stirred into 1 liter of pyridine) are added. The sample is dissolved completely in the acetylation mixture, optionally with stirring and heating. 5 ml of catalyst solution (catalyst solution: 2 g of 4-dimethylaminopyridine are dissolved in 100 ml of pyridine) are added. The reaction vessel is closed and placed into a water bath preheated to 55° C. for 10 minutes while thoroughly mixing. The reaction solution is then admixed with 10 ml of demineralized water, the reaction vessel is again closed and left to react again for 10 minutes in the shaking water bath. The sample is cooled to room temperature (25° C.). Then, 50 ml of 2-propanol and 2 drops of phenolphthalein are added. This solution is titrated with sodium hydroxide solution (sodium hydroxide solution c=0.5 mol/l) (Va). Under the same conditions, but without initial weight of sample, the active value of the acetylation mixture is determined (Vb).

The consumption of the active value determination and of the titration of the sample is used to calculate the OH number (OHN) according to the following formula:

$$O H N = \frac{( Vb - Va ) \cdot c \cdot t \cdot M}{E}$$

OHN=hydroxyl number in mg KOH/g substance
Va=consumption of sodium hydroxide solution in ml during the titration of the sample
Vb consumption of sodium hydroxide solution in ml during the titration of the active value
c=quantitative concentration of the sodium hydroxide solution in mol/l
t=titer of the sodium hydroxide solution
M=molar mass of KOH=56.11 g/mol
E=sample initial weight in g

(Vb−Va) is the amount of sodium hydroxide solution used in ml Which is equivalent to the amount of acetic acid bonded during the above-described acetylation of the sample to be measured.

The amount of composition A in the antiperspirant or deodorant is preferably from 0.1 to 20.0% by weight, particularly preferably from 0.5 to 15.0% by weight, particularly preferably from 2.0 to 13.0% by weight and extraordinarily preferably from 3.0 to 10.0% by weight, based on the total weight of the antiperspirant or deodorant.

Preferably, the antiperspirants and deodorants comprise no aluminum-containing compounds.

In a particularly preferred embodiment of the invention, the antiperspirants and deodorants comprise no further substances active against body odor. In the context of the present invention, further substances active against body odor are understood in particular as meaning compounds which are different to caprylic acid, capric acid, sorbitol, sorbitol caprylates, sorbitol caprates, sorbitan, sorbitan caprylates/caprates, isosorbide and isosorbide caprylates/caprates.

In a further particularly preferred embodiment of the invention, the antiperspirants and deodorants comprise one or more further substances active against body odor.

Preferably, the one or more further substances active against body odor is/are selected from the group consisting of Acorus Gramineus Root/Stem/Luffa Cylindrica Fruit/Camellia Sinensis Leaf Extract, Adipic Acid/Neopentyl Glycol Crosspolymer, Alpinia Uralensis Stalk/Leaf Water, Amber Powder, Ammonium Phenolsulfonate, Ammonium Silver Zeolite, Ammonium Silver Zinc Aluminium Silicate, Benzalkonium Bromide, Benzalkonium Cetyl Phosphate, Benzalkonium Chloride, Benzalkonium Saccharinate, Benzethonium Chloride, Boesenbergia Pandurata Rhizome Extract, Bromochlorophene, Bursera Graveolens Fruit Oil, Butyl Acrylate/Ethyltrimonium Chloride Methacrylate/Styrene Copolymer, t-Butyl Methylphenoxy Phenol, Butyloctanoic Acid, Calcium Magnesium Silicate, Callicarpa Macrophylia Flower Extract, Candida Bornbicola/Glucose/Methyl Rapeseedate Ferment, Capramidoethyl Capramidopropyldimonium Methosultate, Caprylol Gold of Pleasure Amino Acids, Caprylyl Glycol, Castanea Crenata (chestnut) Pellicle Extract, Cetylpyridinium Chloride, Chitosan, Chlorophyllin-Copper Complex, Chlorothymol, Chloroxylenol, Citrus Reticulata (Tangerine) Peel Oil, Cioflucarban, Coix Lacryma-jobi Ma-Yuen Seed/Pueraria Lobata Root/Gandoderma Lucidum (Mushroom) Extract, Colloidal Platinum, Curcuma Heyneana Root Powder, Cyciopentadecanone, Dequalinium Chloride, Dichlorophene, Dichloro-m-Xylenol, DimethiconePEG-15 Crosspolymer, Dimethylbicycloheptyl Ethanone, Dipotassium Capryloyl Glutamate, Disodium Capryloyl Glutamate, Disoclium Dihydroxyethyl Sultosuccinylundecylenate, Domiphen Bromide, Ethylhexylglycerin, Fermented Vegetable, Ferric Chloride, Geranic Acid, Glyceryl Caprylate, Glyceryl Caprylate/Caprate, Hexachlorophene, HexanediolPEG-2 Cocomonium Chloride/TDI Copolymer, Humulus Lupulus (Hops) Cone Extract, Hydrolyzed Cellulose, Hydrolyzed Sasa Veitchii Extract, Ketoglutaric Acid, Hypericum Perforaturn Flower/Twig Extract., Laury Isoquinolinium Bromide, Laurylpyridinium Chloride, Macelignan, Mentha Aquatica Water, Methylbenzethonium Chloride, 2-Methyl 5-Cyclohexylpentanol, Methyl Phenylbutanol, Methyl Undecylenate, Michelia Champaca Flower Oil, Micrococcus/Hydrolyzed Nonfat Milk Ferment, Octadecenedicic Acid, Octanediol, Octenidine HCl, Oligopeptide-10, Oxidized Beta-Glucan, Pandanus Amaryilifolius Leaf Extract, Panduratin A, Pelargonium Graveolens Water, Phenol, Phyllostachys Edulis Stem Extract, Piper Betle Leaf Oil, Piroctonol, Piroctone Olamine, Polyaminopropyl Biguanide Stearate, Potassium Capryloyl Glutamate, Rapeseed Sophorolipids, Rosmarinus Officinalis (Rosemary) Flower Extract, Saccharomyoes/Persimrnon Fruit Juice Ferment Extract, Saccharomycesi/RhodobacteriLactobacillus/Leuconostoc/Streptomyces Griseus/Aspergillus/Bacillus Ferment Filtrate, Sasa Senanensis Leaf Extract, Sasa Senanensis Leaf Powder, Scallop Shell Powder, Shikimic Acid, Silver Citrate, Silver Chloride (and) Titanium Dioxide, Silver Chloride (and) Titanium Dioxide (and) Diethylhexyl Sodium Sulfosuccinate (and) Propylene Glycol, Silver Copper Zeolite, Silver Lactate, Sodium Bicarbonate, Sodium Capryloyi Glutamate, Sodium Phenolsulfonate, Stemmacantha Carthamoides Root Extract, Totarol, Triclocarban, Triclosan, Tricyclodecenyi Propionate, Triethylcitrate, Urginea. Maritima Tuber Extract, Zeolite, Zinc Dimethicone PEG-8 Succinate, Zinc Lactate, Zinc Phenoisulfonate, Zinc Ricinoleate, Zinc Silicate and Zirconium Powder.

Particularly preferably, the one or more further substances active aaainst body odor is or are selected from the group consisting of Caprylyl Glycol, Chitosan, Ethylhexylalycerin, Glyceryl Caprylate, Glyceryl Caprylate/Caprate, Octanediol, Piroctonol, Piroctone, Olamine, Silver Citrate, Silver Chloride (and) Titanium Dioxide, Silver Chloride (and) Titanium Dioxide (and) Diethylhexyi Sodium Suifosuccinate (and) Propylene Glycol, Silver Lactate, Triclosan, Triethylcitrate and Zinc Ricinoleate.

Particularly preferably, the one or more further substances active against body odor is or are selected from the group consisting of Zinc Rioinoleate, Silver Chloride (and) Titanium Dioxide (and) Diethylhexyl Sodium Sulfosuccinate (and) Propylene Glycol, Piroctonol, Piroctone Olamine, Chitosan, Octanedioi, Ethylhexylglycerin, Caprylyl Glycol, Glyceryl Caprylate, Glyceryl Caprylate/Caprate, Silver Citrate, Silver Lactate, Triclosan and Triethylcitrate.

Extraordinarily preferably, the one or more further substances active against body odor is or are selected from the group consisting of Zinc Ricinoleate, Silver Chloride (and) Titanium Dioxide (and) Diethylhexyl Sodium Sulfosuccinate (and) Propylene Glycol (e.g. JM ActiCare®), Piroctonol, Chitosan, Octanediol and Piroctone Olamine.

Very particularly preferably, the one or more further substances active against body odor is or are selected from the group consisting of
a.) mixtures comprising silver chloride, titanium dioxide, diethylhexyl sodium sulfosuccinate and propylene glycol (e.g. JM ActiCare®) and
b.) chitosan, preferably with average molar masses of 10 000 to 100 000 gimol (e.g. Zenvivo® Protect or Zenvivo® Aqua).

The antiperspirants or deodorants are preferably in the form of sticks, roll-ons, fluids, gels, sprays, lotions or creams.

The antiperspirants of deodorants are preferably formulated on an aqueous or aqueous-alcoholic basis or are in the form of emulsions or dispersions. Particularly preferably, they are in the form of emulsions and especially preferably they are in the form of oil-in-water emulsions.

The antiperspirants or deodorants can comprise, as further auxiliaries and additives, all substances usually used customarily for this application, for example oils, waxes, emulsifiers, coemulsifiers, dispersants, surfactants, antifoams, solubilizers, electrolytes, hydroxy acids, stabilizers, polymers, film formers, thickeners, gelling agents, superfatting agents, refatting agents, further antimicrobial active ingredients, biogenic active ingredients, astringents, active substances, sunscreen filters, antioxidants, oxidants, humectants, solvents, colorants, pigments, pearlizing agents, fragrances, pacifiers and/or silicones.

The antiperspirants or deodorants have pH values of preferably 2 to 11, particularly preferably from 4,5 to 8.5 and particularly preferably from 5.0 to 6.5.

The examples below serve to illustrate the invention in more detail, but without limiting it thereto.

### 1) DETERMINATION OF THE REDUCTION IN BODY ODOR

To determine the reduction in body odor, the following base formulations for roll-on deodorants were used.

Base Formulation I:

| Phase | Ingredient | % by weight |
|---|---|---|
| A | Water | ad 100 |
| B | Talc (Luzenac ® 00) | 9.0 |
| C | Ammonium Acryloyldimethyltaurate/Beheneth-25 | 0.6 |
| | Methacrylate Crosspolymer (Aristoflex ® HMB) | 0.1 |
| | Xanthan Gum | |
| D | Squalane | 9.0 |
| E | Preservative | q.s. |

Preparation:
I Add B to A with stirring
II Add C to I and stir until the solution is homogeneous
III Add D to II
IV Add E to III

Base Formulation II:

| Phase | Ingredient | % by weight |
|---|---|---|
| A | Water | ad 100 |
| B | PEG-150 Distearate (Rewopal ® PEG 6000 DS) | 1.0 |
| C | Ceteareth-25 (Genapol ® T 250) | 5.0 |
| | Butylene Glycol | 3.0 |
| | Dicaprylyl Ether (Cetiol ® OE) | 1.0 |
| | Glyceryl Isostearate | 2.0 |
| D | Water | 15.0 |
| | Lactic acid | q.s. |
| E | Preservative | q.s. |

Preparation:
I Add B to A with stirring at 80° C.
II Dissolve C at 80° C. and add I to C with stirring
III Add D to II
IV Add E to III

Various substances active against body odor were incorporated into the base formulations, for example as a further constituent of component D. These were the following substances:

A) Sorbitan/Isosorbide Caprylate X1 of the Following Composition:

| Substance | % by weight |
|---|---|
| Isosorbide monocaprylate | 37.2 |
| Isosorbide dicaprylate | 15.1 |
| Sorbitan monocaprylate | 11.6 |
| Sorbitan dicaprylate | 9.1 |
| Sorbitan tricaprylate | 4.2 |
| Sorbitan tetracaprylate | 0.5 |
| Isosorbide | 15.9 |
| Sorbitan | 5.5 |
| Sorbitol | 0.1 |
| Caprylic acid | 0.8 |

B) Zinc Ricinoleate

C) JM ActiCare® of the Following Composition:

| Substance | % by weight |
|---|---|
| Silver chloride | 2 |
| Titanium dioxide | 8 |
| Diethylhexyl Sodium Sulfosuccinate | max. 20 |
| Propylene glycol | max. 5 |
| Water | min. 65 |

D) Piroctonol

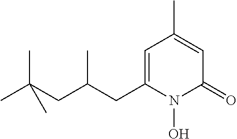 [SMILES: Cc1cc(CC(C)CC(C)(C)C)n(O)c(=O)c1]

E) Zenvivo® Aqua

| Substance | % by weight |
|---|---|
| Poly(1-4)-2-Amino-2-deoxy-β-D-Glucan (Chitosan) | min. 80 |
| with average molar mass of 90 000 g/mol | |
| β-D-Glucan | max. 10 |
| Water | max. 10 |

F) Zenvivo® Protect

| Substance | % by weight |
|---|---|
| Poly(1-4)-2-Amino-2-deoxy-β-D-Glucan (Chitosan) | min. 75 |
| with average molar mass of 15 000 g/mol | |
| β-D-Glucan | max. 15 |
| Water | max. 10 |

To determine the reduction in body odor, the deodorizing action was carried out in the 48-hour sniff test, for which the procedure is as follows:

20 subjects are selected according to the following criteria:
female and male
at least 18 years old
nonsmokers
clinically healthy
readily detectable auxiliary body odor under both armpits

The two armpits of the subjects are preconditioned over several days, i.e. no kind of antiperspirants and deodorants are used and a pH-skin-neutral, unperfumed liquid soap is used.

On the 20 pretreated subjects, in the left or right axilla, after a sing application of 250-300 mg of the base formulation comprising active substances against body odor in the component D, three olfactory experts evaluate the axillary body odor and product scent as to intensity before and after treatment over a total of 48 hours.

The opposite axilla is not treated and serves as a standard for determining the relative intensity decrease in axillary body odor compared to the axilla which has been treated with the base formulation comprising active substances against body odor in component D.

As well as the starting value directly before the single application (after 0 hours) and the end value 48 hours after the single application, the intensity of the axillary body odor and product scent is also evaluated after 24 hours. The intensity of the body odor or perfume scent was assessed by the three olfactory experts according to the following scale:
1=not detectable
2=slightly detectable
3=detectable
4=strongly detectable
5=very strongly detectable

Comparing the intensities of the body odor (or perfume scent) from the treated axilla and the untreated axilla gives, by virtue of the formula below, the relative intensity decrease in axillary body odor as a result of a single application of 250-300 mg of the base formulation comprising active substances against body odor in component D.

$$Intensity decrease in body odor = \frac{( Odor of untreated axilla - odor of treated axilla )}{Odor of untreated axilla} 100 \%$$

### 2) RESULTS FROM THE DETERMINATION FOR REDUCING BODY ODOR

The following results were obtained:

The body odor of the untreated axilla remained constant over the study period.

A perfume scent or intrinsic odor of the test object was not detectable.

Further results are shown in Tables A1-A6.

TABLE A1
Results of experiments for reducing body odor using
sorbitan/isosorbide caprylate X1 after 24 and 48 hours

| | | Result | Result |
|---|---|---|---|
| | | after | after |
| Composition added to base | Base | 24 hours | 48 hours |
| formulation | formulation | [%] | [%] |
| None | I | 0 | 0 |
| 7.6% by weight sorbitan/isosorbide | I | 23 | 21 |
| caprylate X1 | | | |
| None | II | 0 | 0 |
| 1.0% by weight sorbitan/isosorbide | II | 10 | 9 |
| caprylate X1 | | | |
| 4.0% by weight sorbitan/isosorbide | II | 21 | 20 |
| caprylate X1 | | | |

The results in Table Al reveal that the ingredients of the base formulation do not reduce the body odor. The addition of up to 7.6% by weight of sorbitan/isosorbide caprylate X1 reduces the intensity of the body odor over the investigated period of 48 hours following use. It is notable here that the reduction in intensity of the body odor 48 hours after application is surprisingly only slightly below the value 24 hours after application.

TABLE A2
Results of experiments for reducing body odor using
sorbitan/isosorbide caprylate X1, octanediol, zinc
ricinoleate and combination after 24 and 48 hours

| | Result | Result |
|---|---|---|
| | after | after |
| | 24 hours | 48 hours |
| Composition added to base formulation I | [%] | [%] |
| 7.6% by weight octanediol | 18 | 13 |
| 7.6% by weight sorbitan/isosorbide caprylate X1 | 23 | 21 |
| 7.6% by weight octanediol and | 30 | 13 |
| 1.8% by weight zinc ricinoleate | | |
| 7.6% by weight sorbitan/isosorbide caprylate X1 | 28 | 23 |
| and | | |
| 1.8% by weight zinc ricinoleate | | |

The results in Table A2 reveal that both sorbitan/isosorbide caprylate X1 and also octanediol improve the action against body odor both after 24 and after 48 hours. Here, the sorbitan/isosorbide caprylate X1 leads to better results, especially after 48 hours.

Comparing the results for the mixture of octanediol and zinc ricinoleate on the one hand and for the mixture of sorbitan/isosorbide caprylate X1 and zinc ricinoleate on the other hand reveals that the action against body odor after 24 hours is comparable for both mixtures, but the mixture with sorbitan/isosorbide caprylate X1 after 46 hours leads to a significantly better action against body odor.

TABLE A3
Results of experiments for reducing body odor using
sorbitan/isosorbide caprylate X1 and JM
ActiCare ® after 24 and 48 hours

| | Result | Result |
|---|---|---|
| | after | after |
| | 24 hours | 48 hours |
| Composition added to base formulation I | [%] | [%] |
| 0.2% by weight JM ActiCare ® | 21 | 21 |
| 0.2% by weight JM ActiCare ® and | 30 | 25 |
| 7.6% by weight sorbitan/isosorbide caprylate X1 | | |

The results in Table A3 reveal that sorbitan/isosorbide caprylate X1 in antiperspirants and deodorants improves the action of JM ActiCare® as regards the extent and duration of the reduction in body odor.

TABLE A4
Results of experiments for reducing body odor using
sorbitan/isosorbide caprylate X1 and Piroctonol
after 24 and 48 hours

| | Result | Result |
|---|---|---|
| | after | after |
| | 24 hours | 48 hours |
| Composition added to base formulation I | [%] | [%] |
| 0.1% by weight Piroctonol, 5% by weight | 15 | 10 |
| propylene glycol^{[1]} | | |
| 0.1% by weight Piroctonol, 5% by weight | 30 | 21 |
| propylene glycol^{[1]} and | | |
| 7.6% by weight sorbitan/isosorbide caprylate X1 | | |

^{[1]}5% by weight propylene glycol is added as solubilizer for 0.1% by weight piroctonol.

The results of Table A4 reveal that sorbitan/isosorbide caprylate X1 in antiperspirants and deodorant improves the action of piroctonol as regards the extent and duration of reducing body odor.

TABLE A5
Results of experiments for reducing body odor using sorbitan/isosorbide
caprylate X1 and Zenvivo ® Aqua after 24 and 48 hours

| | Result | Result |
|---|---|---|
| | after | after |
| | 24 hours | 48 hours |
| Composition added to base formulation II | [%] | [%] |
| 0.4% by weight Zenvivo ® Aqua | 32 | 26 |
| 1.0% by weight sorbitan/isosorbide caprylate | 41 | 34 |
| X1 + 0.4% by weight Zenvivo ® Aqua | | |

The results in Table AS reveal that sorbitan/isosorbide caprylate X1 in antiperspirants and deodorants improves the action of Zenvivo® Aqua as regards the extent and duration of the reduction in body odor.

TABLE A6
Results of experiments for reducing body odor using sorbitan/isosorbide
caprylate X1 and Zenvivo ® Protect after 24 and 48 hours

| | Result | Result |
|---|---|---|
| | after | after |
| | 24 hours | 48 hours |
| Composition added to base formulation II | [%] | [%] |
| 0.4% by weight Zenvivo ® Protect | 28 | 19 |
| 1.0% by weight sorbitan/isosorbide caprylate | 32 | 25 |
| X1 + 0.4% by weight Zenvivo ® Protect | | |

The results in Table A6 reveal that sorbitan/isosorbide caprylate X.1 in antiperspirants and deodorants improves the action of Zenvivo® Protect as regards the extent and duration of the reduction in body odor.

### 3) FORMULATION EXAMPLES

The use according to the invention can take place for example in the following formulations.

### Formulation Example 1

### Roll-On Deodorant

| Phase | Ingredient | % by weight |
|---|---|---|
| A | Hydroxyethyl Cellulose (Tylose ® H 10000 G4) | 0.7 |
| B | Water | ad 100 |
| C | Piroctone Olamine (Octopirox ®) | 0.2 |
| D | Ethanol | 30.0 |
| | Sorbitan/isosorbide caprylate X1 | 8.0 |
| E | Propylene Glycol | 5.0 |
| | PEG-40 Hydrogenated Castor Oil (Emulsogen ® | 0.5 |
| | HCO 040) | |
| F | Citric Acid (pH 5.0-5.5) | q.s. |

Preparation:
I Add A to B with continuous stirring until the solution is homogeneous.
II Dissolve C in D and add the components from E.
III Add II to I
IV Adjust the pH using F.

### Formulation Example 2

### Deodorant Stick

| Phase | Ingredient | % by weight |
|---|---|---|
| A | Piroctone Olamine (Octopirox ®) | 0.1 |
| | Sorbitan/isosorbide caprylate X1 | 4.0 |
| | 1,3-Butanediol | 30.0 |
| | Propylene glycol | 27.0 |
| | Isosteareth-20 (Rewoderm ® 66E) | 4.0 |
| | Steareth-2 (Genapol ® HS 020) | 1.0 |
| B | Polyglycol 1500 (PEG-32) | 5.0 |
| C | Water | ad 100 |
| D | Sodium stearate | 6.0 |

Preparation:
I Mix the components from A, heat to approx. 50° C. and homogenize with stirring.
Dissolve B in C with stirring and heat to approx. 50° C.
III Mix I and II, add D and dissolve D with stirring and heat until the solution is clear.
IV Pour III into deodorant stick housing and leave the formulation to cool.

### Formulation Example 3

### Deodorant Gel

| Phase | Ingredient | % by weight |
|---|---|---|
| A | PEG-40 Hydrogenated Castor Oil (Emulsogen ® | 1.0 |
| | HCO 040) | |
| | Ethanol | 25.0 |
| | Piroctone Olamine (Octopirox ®) | 0.1 |
| | Sorbitan/isosorbide caprylate X1 | 3.0 |
| B | Propylene glycol | 20.0 |
| | Diisopropyl Adipate (Isoadipate © 660014) | 1.0 |
| | Water | ad 100 |
| C | Citric acid | q.s. |
| D | Ammonium Acryloyldimethyltaurate/VP | 1.3 |
| | copolymer (Aristoflex ® AVC) | |

Preparation:
I Mix the components from A until the solution is clear.
II Add B with stirring to I.
III Adjust the pH of II to 5.5-6.0 using C.
IV. Add D to III with stirring.

### Formulation Example 4

### Antiperspirant in the Form of a Foot Spray

| Phase | Ingredient | % by weight |
|---|---|---|
| A | Piroctone Olamine (Octopirox ®) | 0.3 |
| | Menthol | 0.1 |
| | Sorbitan/isosorbide caprylate X1 | 2.0 |
| B | Ethanol | 55.0 |
| | Propylene glycol | 5.0 |
| C | Allantoin | 0.1 |
| | Panthenol | 0.5 |
| | Water | ad 100 |
| D | Citric acid | q.s. |

Preparation:
I Dissolve A in B.
II Add heated C to I with stirring.
III Adjust the pH of II to 5.5-6.0 using D.

### Formulation Example 5

### Deodorant in the Form of a Foot Cream

| Phase | Ingredient | % by weight |
|---|---|---|
| A | Piroctone Olamine (Octopirox ®) | 0.2 |
| | Propylene glycol | 2.0 |
| B | Triceteareth-4 Phosphate (Hostaphat ® KW 340 D) | 2.5 |
| | Sorbitan/isosorbide caprylate X1 | 5.0 |
| | Caprylyl Methicone (SilCare ® Silicone 41M15) | 1.0 |
| | PEG-4 Polyglyceryl-2 Stearate (Hostacerin ® | 1.5 |
| | DGSB) | |
| | Cetearyl alcohol | 2.0 |
| | Caprylic/capric triglyceride (Velsan ® CCT) | 4.0 |
| C | Ammonium Acryloyldimethyltaurate/VP copolymer | 0.8 |
| | (Aristoflex ® AVC) | |
| D | Water | ad 100 |
| E | Propylene glycol | 2.0 |
| | Camphor | 0.1 |
| | Menthol | 0.2 |
| F | Citric Acid | q.s. |

Preparation:
I Mix the components of A and heat with stirring until the solution is clear.
II Add the components of B to I and heat with stirring to 60° C.
III Add C to II.
IV Heat D to approx. 60° C. and add it to III with stirring.
V Add the heated mixture of the components from E to IV.
VI Adjust the pH of V to 5.5-6.0 using F.

In formulation examples 1-5, instead of piroctone olamine, it is also possible to incorporate zinc ricinoleate, JM ActiCare®, Piroctonol, Chitosan (Zenvivo® Aqua, Zenvivo® Protect), octanediol, ethylhexyl glycerol, caprylyl glycol, glyceryl caprylate, glyceryl caprylate/caprate, silver citrate, silver lactate, triclosan and triethyl citrate as additional substance active against body odor as well as sorbitan/isosorbide caprylate X1.

In formulation examples 1-5, instead of sorbitan/isosorbide caprylate X1, it is also possible to incorporate sorbitan/isosorbide caprylate X2, isosorbide caprylate X3, sorbitan/isosorbide caprate X4, sorbitan/isosorbide caprate X5, isosorbide caprate X6, sorbitan/isosorbide caprylate/caprate X7, sorbitan/isosorbide caprylate/caprate X8 and isosorbide caprylate/caprate X9.

G) Sorbitan/Isosorbide Caprylate X2 of the Following Composition:

| Substance | % by weight |
|---|---|
| Isosorbide monocaprylate | 18.0 |
| Isosorbide dicaprylate | 5.0 |
| Sorbitan monocaprylate | 22.1 |
| Sorbitan dicaprylate | 21.0 |
| Sorbitan tricaprylate | 8.6 |
| Sorbitan tetracaprylate | 1.0 |
| Isosorbide | 15.8 |
| Sorbitan | 8.1 |
| Sorbitol | 0.1 |
| Caprylic acid | 0.3 |

H) Isosorbide Caprylate X3 of the Following Composition:

| Substance | % by weight |
|---|---|
| Isosorbide monocaprylate | 50.9 |
| Isosorbide dicaprylate | 30.6 |
| Isosorbide | 18.1 |
| Caprylic acid | 0.4 |

K) Sorbitan/Isosorbide Caprate X4 of the Following Composition:

| Substance | % by weight |
|---|---|
| Isosorbide monocaprate | 34.2 |
| Isosorbide dicaprate | 13.1 |
| Sorbitan monocaprate | 14.6 |
| Sorbitan dicaprate | 10.3 |
| Sorbitan tricaprate | 5.0 |
| Sorbitan tetracaprate | 0.5 |
| Isosorbide | 15.9 |
| Sorbitan | 5.5 |
| Sorbitol | 0.1 |
| Capric acid | 0.8 |

L) Sorbitan/Isosorbide Caprate X5 of the Following Composition:

| Substance | % by weight |
|---|---|
| Isosorbide monocaprate | 16.0 |
| Isosorbide dicaprate | 5.0 |
| Sorbitan monocaprate | 24.1 |
| Sorbitan dicaprate | 21.0 |
| Sorbitan tricaprate | 8.6 |
| Sorbitan tetracaprate | 1.0 |
| Isosorbide | 15.8 |
| Sorbitan | 8.1 |
| Sorbitol | 0.1 |
| Capric acid | 0.3 |

M) isosorbide Caprate X6 of the Following Composition:

| Substance | % by weight |
|---|---|
| Isosorbide monocaprate | 53.4 |
| Isosorbide dicaprate | 28.6 |
| Isosorbide | 17.6 |
| Capric acid | 0.4 |

N) Sorbitan/Isosorbide Caprylate/Caprate X7 of the Following Composition:

| Substance | % by weight |
|---|---|
| Isosorbide monocaprylate | 18.9 |
| Isosorbide monocaprate | 18.3 |
| Isosorbide dicaprylate/caprate^{a)} | 15.1 |
| Sorbitan monocaprylate | 6.0 |
| Sorbitan monocaprate | 5.6 |
| Sorbitan dicaprylate/caprate^{a)} | 9.1 |
| Sorbitan tricaprylate/caprate^{a)} | 4.2 |
| Sorbitan tetracaprylate/caprate^{a)} | 0.5 |
| Isosorbide | 15.9 |
| Sorbitan | 5.5 |
| Sorbitol | 0.1 |
| Caprylic acid | 0.4 |
| Capric acid | 0.4 |

^{a)}The terms such as “isosorbide dicaprylate/caprate” given for the composition X7 mean that either pure caprylates or pure caprates as well as mixtures of caprylates and caprates may be present on account of the plurality of ester bonds in one molecule.

F) Sorbitan/Isosorbide Caprylate/Caprate X8 of the Following Composition:

| Substance | % by weight |
|---|---|
| Isosorbide monocaprylate | 9.1 |
| Isosorbide monocaprate | 8.9 |
| Isosorbide dicaprylate/caprate^{a)} | 5.0 |
| Sorbitan monocaprylate | 11.2 |
| Sorbitan monocaprate | 10.9 |
| Sorbitan dicaprylate/caprate^{a)} | 21.0 |
| Sorbitan tricaprylate/caprate^{a)} | 8.6 |
| Sorbitan tetracaprylate/caprate^{a)} | 1.0 |
| Isosorbide | 15.8 |
| Sorbitan | 7.9 |
| Sorbitol | 0.1 |
| Caprylic acid | 0.3 |
| Capric acid | 0.2 |

^{a)}see explanation for N) sorbitan/isosorbide caprylate/caprate X7

R) Isosorbide Caprylate/Caprate X9 of he Following Composition:

| Substance | % by weight |
|---|---|
| Isosorbide monocaprylate | 25.9 |
| Isosorbide monocaprate | 25.0 |
| Isosorbide dicaprylate/caprate^{a)} | 30.6 |
| Isosorbide | 18.1 |
| Caprylic acid | 0.2 |
| Capric acid | 0.2 |

^{a)}see explanation for N) sorbitan/isosorbide caprylate/caprate X7

1. A process for improving the action of an antiperspirant and/or a deodorant comprising the step of adding a composition comprising at least one isosorbide caprylate/caprate (composition A) to the antiperspirant and/or deodorant, wherein the OH number of the mixture present in the composition A of the at least one isosorbide caprylate/caprate is less than or equal to 260.

2. The process as claimed in claim 1, wherein the composition A further comprises at least one sorbitan caprylate/caprate.

3. The process as claimed in claim 1, wherein the effect of the antiperspirant and deodorant improved is the extent of the reduction in body odor.

4. The process as claimed in claim 1, wherein the effect of the antiperspirant and deodorant improved is the duration of the reduction in body odor.

5. The process as claimed in claim 1, wherein the composition A further comprises, at least one compound selected from the group consisting of caprylic acid, capric acid, sorbitol, sorbitol caprylates, sorbitol caprates, sorbitan and isosorbide; and optionally one or more sorbitan caprylates/caprates.

6. The process as claimed in claim 2, wherein the total amount in the composition A of the at least one isosorbide caprylate/caprate and the optionally at least one sorbitan caprylate/caprate additionally present in the composition A is at least 50.0% by weight, based on the total weight of the composition A.

7. The process as claimed in claim 1, wherein the composition A comprises isosorbide monocaprylate/caprate.

8. The process as claimed in claim 7, wherein the composition A further comprises sorbitan monocaprylate/caprate alongside isosorbide monocaprylate/caprate.

9. The process as claimed in claim 7, wherein the total amount in the composition A of isosorbide monocaprylate/caprate and the sorbitan monocaprylate/caprate optionally additionally present in the composition A is at least 30.0% by weight, based on the total weight of the composition A.

10. The process as claimed in claim 8, wherein the composition A further comprises sorbitan monocaprylate/caprate alongside isosorbide monocaprylate/caprate and the weight ratio of sorbitan monocaprylate/caprate to isosorbide monocaprylate/caprate in the composition A is from 20:1 to 1:100.

11. The process as claimed in claim 1, wherein the amount of composition A in the antiperspirant or deodorant is from 0.1 to 20.0% by weight, based on the total weight of the antiperspirant or deodorant.

12. The process as claimed in claim 1, wherein the antiperspirant and/or deodorant comprise no aluminum-containing compounds.

13. The process as claimed in claim 1, wherein the antiperspirant and/or deodorant comprise no further substances active against body odor.

14. The process as claimed in claim 1, wherein the antiperspirant and/or deodorant further comprise at least one substance active against body odor.

15. The process as claimed in claim 14, wherein the at least one substance active against body odor is selected from the group consisting of Caprylyl Glycol, Chitosan, Ethylhexylglycerin, Glyceryl Caprylate, Glyceryl Caprylate/Caprate, Octanediol, Piroctonol, Piroctone Olamine, Silver Citrate, Silver Chloride (and) Titanium Dioxide, Silver Chloride (and) Titanium Dioxide (and) Diethyihexyl Sodium Sulfosuccinate (and) Propylene Glycol, Silver Lactate, Triclosan, Triethylcitrate and Zinc Ricinoleate.
